(19) United States
(12) Patent Application Publication
(10) Pub. No.: US 2010/0262181 A1
(43) Pub. Date: Oct. 14, 2010

(54) ENDO PSI AND CABINET THEREFOR

(76) Inventors: Seung Wook CHOI, Seongnam-si (KR); Jong Seok Won, Yongin-si (KR)

Correspondence Address: BIRCH STEWART KOLASCH & BIRCH, PO BOX 747, FALLS CHURCH, VA, 22040-0747, US

(21) Appl. No.: 12/755,042

(22) Filed: Apr. 6, 2010

(30) Foreign Application Priority Data
Apr. 8, 2009 (KR) 10-2009-0030151
Oct. 20, 2009 (KR) 10-2009-0099519
Feb. 19, 2010 (KR) 10-2010-0014968

(51) Int. Cl. A61B 17/28

(52) U.S. Cl. 606/206; 606/205

(57) ABSTRACT

An aspect of the invention provides a PSI (purse-string instrument) for laparoscopic surgery that includes: a first jaw; a second jaw configured to clamp a suture site in cooperation with the first jaw; a first rod, which is coupled to one end of the first jaw and configured to rotate the first jaw such that the suture site is clamped, and which is held inside a shaft; and a second rod, which is coupled to one end of the second jaw and configured to rotate the second jaw such that the second jaw faces an extending direction of the shaft. The PSI can be inserted using the incision made beforehand in the surgical site during laparoscopic surgery and thus does not require making additional cuts in the patient's body or disassembling/reassembling the instrument when inserting the instrument into the abdominal cavity.

### CROSS-REFERENCE TO RELATED APPLICATIONS

This application claims the benefit of Korean Patent Application No. 10-2009-0030151 filed with the Korean Intellectual Property Office on Apr. 8, 2009, Korean Patent Application No. 10-2009-0099519 filed with the Korean Intellectual Property Office on Oct. 20, 2009, and Korean Patent Application No. 10-2010-0014968 filed with the Korean Intellectual Property Office on Feb. 19, 2010, the disclosures of which are incorporated herein by reference in their entirety.

### BACKGROUND

The present invention relates to a surgical apparatus, more particularly to a PSI (purse-string instrument) for laparoscopic surgery, a method of using the PSI, and a cabinet for using the PSI.

In the field of medicine, surgery refers to a procedure in which a medical apparatus is used to make a cut or an incision in or otherwise manipulate a patient's skin, mucosa, or other tissue, to treat a pathological condition. A surgical procedure such as a laparotomy, etc., in which the skin is cut open and an internal organ, etc., is treated, reconstructed, or excised, may entail problems of blood loss, side effects, pain, and scars. Thus, methods of surgery that involve making an incision in the skin and inserting only a medical apparatus, such as a laparoscope, a surgical instrument, and a microscope, for example, are currently regarded as popular alternatives.

The surgical PSI (purse-string instrument), for suturing the surgical site of an organ after surgery, has long been taught in various ways in the field of surgery as a means for attaching and healing tissues. In the related art, there have been several types of technology developed related to intraluminal or circular PSI's, to provide anastomosis for surgical procedures.

FIG. 1 is a front view of a PSI for laparoscopic surgery according to the related art. Illustrated in FIG. 1 are a first jaw 110, a second jaw 120, a shaft 130, a grip 140, a rod 150, detents 155, a stopper 160, and a spring 165. The jaws may include a first jaw 110 and a second jaw 120 to grab a suture site. The second jaw 120 may be connected with the rod 150 to move closer to or further from the first jaw 110. The user may hold the grip 140, push the rod 150 along the direction of the shaft 130 to grab the suture site, and then secure the rod 150 to the stopper using the detents 155. Afterwards, the user may insert a needle (not shown) through a channel that passes through both ends of the first jaw 110 and second jaw 120, to suture the surgical site.

According to the related art, however, the size of the jaws may be greater than the thickness of the shaft 130. The jaws may not be able to pass through the incision used for laparoscopic surgery, and the incision may have to be widened, resulting in more surgery scars.

Also, according to the related art, the jaws may be bent by a fixed angle and thus may not be able to pass through the trocar used during laparoscopic surgery. Hence, a procedure according to the related art may entail making a greater incision in the surgical site or performing unnecessary processes such as disassembling the PSI, inserting the shaft through the trocar, and then reassembling the PSI to access the surgical site.

Moreover, according to the related art, inserting the needle through the jaws may require pushing the needle through the channel, as described above, while holding the needle using a laparoscope instrument. As this involves a certain amount of force, even a slight crook in the needle may cause the needle to deviate from the channel path inside, preventing the needle from being inserted. As such, the user may have to be careful to see that the needle is not crooked while being inserted into the channel.

The information in the related art described above was obtained by the inventors for the purpose of developing the present invention or was obtained during the process of developing the invention. As such, it is to be appreciated that this information did not necessarily belong to the public domain before the patent filing date of the invention.

### SUMMARY

An aspect of the invention is to provide a PSI for laparoscopic surgery that can be inserted using the incision made beforehand in the surgical site during laparoscopic surgery and thus does not require making additional cuts in the patient's body or disassembling/reassembling the instrument when inserting the instrument into the abdominal cavity.

Another aspect of the invention is to provide a PSI for laparoscopic surgery, as well as a cabinet for using the PSI, with which the needle can be inserted easily during suturing.

One aspect of the invention provides a PSI for laparoscopic surgery that includes: a first jaw; a second jaw configured to clamp a suture site in cooperation with the first jaw; a first rod, which is coupled to one end of the first jaw and configured to rotate the first jaw such that the suture site is clamped, and which is held inside a shaft; and a second rod, which is coupled to one end of the second jaw and configured to rotate the second jaw such that the second jaw faces an extending direction of the shaft.

The first rod can be coupled by a wire with the first jaw, and the first jaw and the second jaw can be inserted inside the shaft by a linear movement of the second rod.

Another aspect of the invention provides a PSI for laparoscopic surgery that includes: a set of jaws including a first jaw and a second jaw configured to clamp a suture site; a scissor link coupled to one end of each of the first jaw and the second jaw for adjusting a distance between the first jaw and the second jaw; and a first rod, which is coupled to one end of the scissor link and configured to control the scissor link such that the suture site is clamped.

This embodiment can further include a shaft, which may hold the first rod and which may be equipped on one end with a bending part that is capable of bending. The embodiment can further include a second rod coupled to the bending part and configured to adjust a bending angle of the bending part. The second rod can be coupled with the bending part by a wire that applies a tensional force on the bending part.

The second rod can be held inside the first rod. The embodiment can further include a stopper, which may be coupled to one end of the shaft and configured to be caught on a detent formed on one end of the first rod such that the first rod is stopped. Also, the first rod and the second rod can be configured to move linearly, and a portion of the first rod can be formed as a spring.

Yet another embodiment of the invention provides a PSI for laparoscopic surgery that includes: a first jaw configured to be movable and rotatable by way of a first hinge axis; a second jaw, which may be configured to clamp a suture site in cooperation with the first jaw and be movable and rotatable by way of a second hinge axis; a shaft holding the first jaw and the second jaw; and a main rod coupled to the second hinge axis and configured to move the second hinge axis inside the shaft.

This embodiment can further include a rotation member coupled with the first jaw and configured to apply a rotational force on the first jaw. The rotation member can be an elastic wire or a spring, and the first hinge axis and the second hinge axis can provide rotational forces in different directions.

This embodiment can also include an elastic member, which may be coupled to one or more of the first hinge axis and the second hinge axis to rotate the first jaw or the second jaw. Here, the elastic member can be any one or more of a V-spring, a flat spring, and a helical spring.

The embodiment can further include a first rotation rod, which may have one end coupled to the first jaw, and which may be held inside the shaft and be configured to rotate the first jaw; and a second rotation rod, which may have one end coupled to the second jaw, and which may be held inside the shaft and be configured to rotate the second jaw.

The embodiment can also include an elastic member that is coupled to one side of the second rotation rod and configured to apply an elastic force on the second rotation rod so as to rotate the second jaw in one direction.

Here, the shaft can be shaped as a hollow tube, and the first rotation rod and the second rotation rod can be held inside the shaft. A trench can be formed in the main rod, extending along an axial direction of the main rod, and the first rotation rod and the second rotation rod can be held in the trench.

The other end of the first rotation rod can be coupled to a lever protruding from a side surface of the shaft. An indentation can be formed in one end of the main rod, and the other end of the second rotation rod can be configured to fit into the indentation.

The embodiment can further include a stopper coupled to one end of the shaft that is configured to be caught on a detent formed on one end of the first rod such that the first rod is stopped.

Also, the main rod can be made to move in linear movements, and a portion of the main rod can be formed as a spring. A channel formed through both ends of one or more of the first jaw and the second jaw to receive a needle inserted therein can have its opening shaped as a funnel, and a diameter of the jaws, when the first jaw and the second jaw are clenched, can be made smaller than a diameter of the shaft.

Another embodiment of the invention provides a cabinet for a PSI for laparoscopic surgery, for inserting a needle into a jaw that has a channel passing through both ends thereof. The cabinet includes: a frame, in which the jaw is inserted; and a support part, which is coupled to the frame and configured to support the needle in correspondence with an opening of the channel when one end of the jaw is inserted.

This embodiment can further include a protruding part, which may be coupled to the frame, and which may be configured to be inserted in correspondence with a trench formed in the jaw. The number of support parts can correspond with the number of channels formed in the jaw, and one or more sides of the cabinet can be open.

Yet another embodiment of the invention provides a method of using a PSI for laparoscopic surgery that includes: inserting a set of jaws, which includes a first jaw and a second jaw that clamp a suture site, inside the abdominal cavity of a patient while keeping the jaws parallel to the shaft; rotating the jaws to a particular angle with respect to the shaft; clamping the suture site with the jaws; and passing a needle through a channel formed in the jaws to suture the suture site.

This embodiment can further include a protruding part, which may be coupled to the frame, and which may be configured to be inserted in correspondence with a trench formed in the jaw. In this case, the trench formed in the jaw can be parallel to an extending direction of the jaw, and the trench formed in the jaw can include an angled part that slants by a particular slope with respect to an extending direction of the jaw.

In this case, the slope can be between 0 and 90 degrees, the angled part can be formed at an end portion of the trench, and there can be a multiple number of angled parts.

Also, the number of support parts can correspond with the number of channels formed in the jaw, and one or more sides of the cabinet, other than the opening through which the jaw is inserted, can be open.

Here, the support part can be located at a surface opposite the opening through which the jaw is inserted, and the support can be made of a flexible material.

The support part can be shaped as any one of a cylinder, a cylindroid, and a prism. In a side of the support part, a side gap can be formed that allows the inserted needle to be detached from the support part when a force is applied in a direction orthogonal to an extending direction of the needle.

Another aspect of the invention provides a PSI for laparoscopic surgery in which a needle can be inserted using the cabinet described above. The PSI includes: a first jaw, and a second jaw that clamps a suture site in cooperation with the first jaw, where a channel for inserting the needle is formed in any one or more of the first jaw and the second jaw.

A PSI for laparoscopic surgery according to an embodiment of the invention can be inserted using the incision made beforehand in the surgical site during laparoscopic surgery and thus does not require making additional cuts in the patient's body or disassembling/reassembling the instrument when inserting the instrument into the abdominal cavity. Also, a cabinet used for the PSI for laparoscopic surgery according to an embodiment of the invention makes it possible to readily insert a needle during suturing.

Additional aspects, features, and advantages, other than those described above, will be obvious from the claims and written description below.

### BRIEF DESCRIPTION OF THE DRAWINGS

FIG. 1 is a front view of a PSI for laparoscopic surgery according to the related art.

FIG. 2 is a first front view of a PSI for laparoscopic surgery according to a first disclosed embodiment of the invention.

FIG. 3 is a second front view of a PSI for laparoscopic surgery according to a first disclosed embodiment of the invention.

FIG. 4 is a third front view of a PSI for laparoscopic surgery according to a first disclosed embodiment of the invention.

FIG. 5 is a partial side view of a PSI for laparoscopic surgery according to a first disclosed embodiment of the invention.

FIG. 6 is a partial perspective view of a jaw on a PSI for laparoscopic surgery according to a first disclosed embodiment of the invention.

FIG. 7 is a front view of a PSI for laparoscopic surgery according to a second disclosed embodiment of the invention.

FIG. 8 is a first front view of a PSI for laparoscopic surgery according to a third disclosed embodiment of the invention.

FIG. 9 is a second front view of a PSI for laparoscopic surgery according to a third disclosed embodiment of the invention.

FIG. 10 is a third front view of a PSI for laparoscopic surgery according to a third disclosed embodiment of the invention.

FIG. 11 is a partial magnified view of a PSI for laparoscopic surgery according to an embodiment of the invention.

FIG. 12 is a partial magnified view of a PSI for laparoscopic surgery according to another embodiment of the invention.

FIG. 13 is a front view of a PSI for laparoscopic surgery according to a fourth disclosed embodiment of the invention.

FIG. 14 is a front view of a PSI for laparoscopic surgery according to a fifth disclosed embodiment of the invention.

FIG. 15A and FIG. 15B are cross-sectional views of PSI's for laparoscopic surgery according to an embodiment of the invention.

FIG. 16A and FIG. 16B are perspective views of cabinets for PSI's for laparoscopic surgery according to another embodiment of the invention.

FIG. 17A and FIG. 17B are perspective views of cabinets for PSI's for laparoscopic surgery according to yet another embodiment of the invention.

FIG. 18 is a perspective view of a PSI for laparoscopic surgery according to an eighth disclosed embodiment of the invention.

FIG. 19A is a perspective view of a cabinet for a PSI for laparoscopic surgery according to an eighth disclosed embodiment of the invention.

FIG. 19B is a partial bottom view of a cabinet for a PSI for laparoscopic surgery according to an eighth disclosed embodiment of the invention.

FIG. 20A, FIG. 20B, FIG. 20C, and FIG. 20D illustrate a process of inserting a needle in a PSI for laparoscopic surgery according to an eighth disclosed embodiment of the invention.

FIG. 21A is a partial side view of a PSI for laparoscopic surgery according to a ninth disclosed embodiment of the invention.

FIG. 21B is a partial side view of a PSI for laparoscopic surgery according to a tenth disclosed embodiment of the invention.

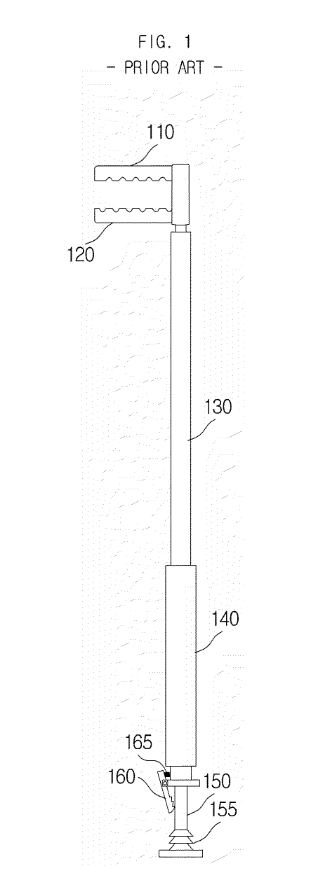

### DETAILED DESCRIPTION

As the present invention allows for various changes and numerous embodiments, particular embodiments will be illustrated in the drawings and described in detail in the written description. However, this is not intended to limit the invention to particular modes of practice, and it is to be appreciated that all changes, equivalents, and substitutes that do not depart from the spirit and technical scope of the invention are encompassed in the present invention.

While terms including ordinal numbers, such as “first” and “second,” etc., may be used to describe various components, such components are not limited to the above terms. The above terms are used only to distinguish one component from another.

When a component is said to be “connected to” or “accessing” another component, it is to be appreciated that the two components can be directly connected to or directly accessing each other but can also include one or more other components in-between.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification.

Unless defined otherwise, all terms used herein, including technical or scientific terms, have the same meanings as those generally understood by those with ordinary knowledge in the field of art to which the invention belongs.

Also, in providing descriptions referring to the accompanying drawings, those components that are the same or are in correspondence are rendered the same reference numeral regardless of the figure number, and redundant descriptions are omitted. In the written description, certain detailed explanations of related art are omitted, when it is deemed that they may unnecessarily obscure the essence of the invention.

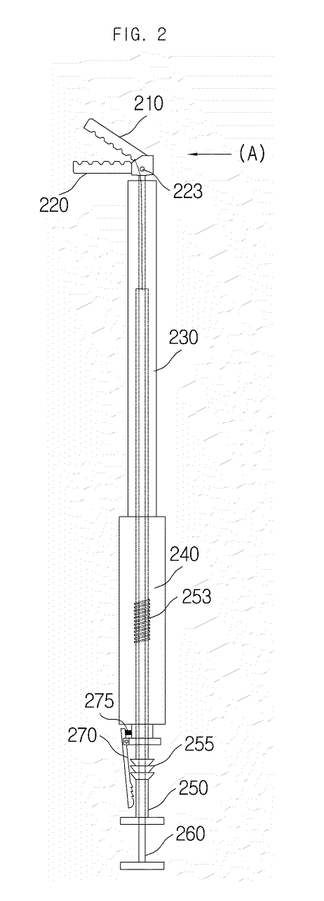

FIG. 2 through FIG. 4 are front views of a PSI for laparoscopic surgery according to a first disclosed embodiment of the invention, for various modes of use. Illustrated in FIGS. 2 to 4 are a first jaw 210, a second jaw 220, a hinge axis 223, a shaft 230, a grip 240, a first rod 250, detents 255, a second rod 260, a stopper 270, and a spring 275.

A feature of this embodiment is that the first jaw 210 and the second jaw 220, which cooperate to clamp a suture site, are able to rotate towards the lengthwise direction in which the shaft 230 is extended, or towards a different direction in correspondence to a user manipulation. That is, the set of jaws, including the first jaw 210 and second jaw 220, can be made to move and rotate, so that the jaws may point towards the lengthwise direction in which the shaft 230 is extended, when being inserted into the abdominal cavity, but point towards a direction forming a particular angle with the shaft 230, for example in a perpendicular direction, when actually suturing the suture site. Also, when an instrument according to this embodiment is being inserted into or withdrawn from the abdominal cavity through a surgical trocar, the inner wall of the surgical trocar can cause the jaws to point in the lengthwise direction in which the shaft 230 is extended, and after the jaws are inserted in the abdominal cavity, a particular rotational force can cause the jaws to form a particular angle with the shaft 230.

The diameter of the jaws, when the first jaw 210 and second jaw 220 are clenched together, can be smaller than the diameter of the shaft 230. In this case, the jaws can be inserted into the abdominal cavity as long as the shaft 230 can be inserted, and therefore the entire instrument can be inserted without having to further increase the cuts. Moreover, in this case, the jaws can be held inside the shaft 230, allowing for easier insertion and storage.

The first jaw 210 and second jaw 220 can include teeth that interlock with one another, so that the suture site may be positioned in-between and be stitched by a needle inserted through a channel that passes through both ends of the first jaw 210 and/or second jaw 220.

The distance between the first jaw 210 and second jaw 220 may be adjusted in order to clamp the suture site. The first rod 250 may be coupled to one end of the first jaw 210 adjacent to the hinge axis 223, and when moved, may rotate the first jaw 210 counter-clockwise about the hinge axis 223 such that the first jaw 210 and second jaw 220 clench together.

To this end, the first jaw 210 can be coupled with the first rod 250 directly or coupled by way of a coupling element such as a wire, etc. For example, if a wire has one end coupled to the first rod 250 and the other end coupled to the first jaw 210 askew of the hinge axis 223, then pulling the first rod 250 downward may cause the wire to transfer a rotational force onto the first jaw 210. If this rotational force is one that effects a counter-clockwise rotation about the hinge axis 223, then the first jaw 210 may rotate in the counter-clockwise direction to clench with the second jaw 220. In order to create such a rotational force, the other end of the wire can be coupled with the first jaw 210 at the left side of the hinge axis 223 in FIG. 2.

A portion of the first rod 250, for clamping the suture site with the first jaw 210 and second jaw 220, can be formed as a spring 253. That is, if a portion of the first rod 250 is formed as a spring 253, then the spring 253 may form a buffer to prevent damage to the suture site when the thickness of the suture site is varied, and also, a constant pressure can be maintained for clamping. The location where the spring 253 is formed can be in a middle region or an arbitrary region of the first rod 250.

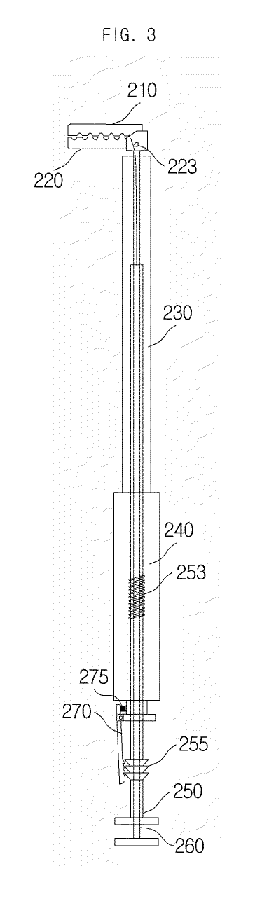

An example in which the first jaw 210 and second jaw 220 are clenched together is illustrated in FIG. 3. In the example shown in FIG. 3, the first rod 250 is pulled downwards, and the detents 255 formed on the first rod 250 are secured by the stopper 270.

An elastic member, such as a spring 275, can apply a rotational force on the stopper 270, which may catch onto the detent 255 and secure the first rod 250, stopping the movement of the first rod 250. That is, the elastic member may push the one end of the stopper 270, to which the elastic member is coupled, further from the shaft 230, and as a result, the other end of the stopper 270 may be forced closer to the first rod 250. Thus, the stopper 270 may secure the first rod 250 using the force of the elastic member.

The inclination of the stopper 270 and the detent 255 can be determined such that the first jaw 210 and second jaw 220 maintains their clamp on the suture site. In the example shown in FIG. 3, since the first rod 250 has moved down to rotate the first jaw 210, the stopper 270 and the detent 255 may be formed with an inclination that prevents the first rod 250 from moving upward, in order to maintain this state.

Also, when the first rod 250 is moved upward, the first jaw 210 may receive a rotational force about the hinge axis 223 in a clockwise direction, so that the jaws may again return to an unclosed state, as in the example shown in FIG. 2. Various embodiments can be applied to the invention to achieve this. For example, an elastic member (such as a spring) can be inserted in the hinge axis 223 that rotates the first jaw 210 clockwise about the hinge axis 223.

Thus, the user is able to close or unclose the jaws by linearly moving the first rod 250 along the lengthwise direction in which the shaft 230 is extended. In this case, in order to determine the maximum angle formed by the first jaw 210 and second jaw 220, a protruding detent (not shown) can be formed along the direction of rotation of the first jaw 210.

Also, the second rod 260 can move the first jaw 210, the second jaw 220, and the hinge axis 223, to alter the lengthwise direction of the first jaw 210 and second jaw 220 to the lengthwise direction of the shaft 230, and furthermore hold these inside the shaft 230. The second rod 260 can be held inside the first rod 250. The second rod 260 can also undergo linear movements to perform the functions described above.

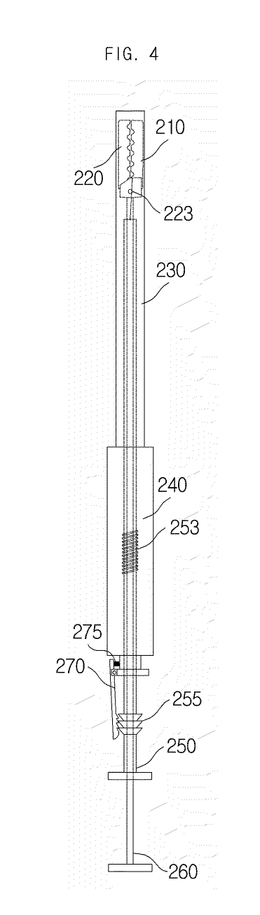

An example in which the first jaw 210, second jaw 220, and hinge axis 223 are held inside the shaft 230 is illustrated in FIG. 4.

In the example shown in FIG. 4, the second rod 260, which is coupled with the first jaw 210, second jaw 220, and hinge axis 223, is pulled downwards, and correspondingly, the first jaw 210, second jaw 220, and hinge axis 223 are held inside the shaft 230. While the coupling relations between the second rod 260 and the first jaw 210, second jaw 220, and hinge axis 223 are not illustrated, these can include direct connections or coupling by wires.

Of course, various methods for adjusting the distance between the first jaw 210 and second jaw 220 can be applied to the invention. For example, while the foregoing descriptions illustrate a case in which the first jaw 210 and second jaw 220 rotate about the same hinge axis 223, the first jaw 210 and the second jaw 220 can be made to clench together by rotating about different, multiple hinge axes.

Also, while the foregoing descriptions illustrate a case in which the first jaw 210 and second jaw 220 clench together by rotating about the hinge axis 223, according to another embodiment, the distance between the first jaw 210 and second jaw 220 can be adjusted while keeping the lengthwise directions of the first jaw 210 and second jaw 220 parallel to each other. For example, the first jaw 210 can be coupled to a hinge axis equipped on the shaft 230, while the second jaw 220 can be coupled to a hinge axis provided on one end of the first rod 250, so that the second jaw 220 may be moved by a movement of the first rod 250 and clench with the first jaw 210 while keeping parallel to the first jaw 210.

A method of using a PSI for laparoscopic surgery according to this embodiment can include, first, inserting the jaws, which include the first jaw 210 and second jaw 220 for clamping the suture site, into the abdominal cavity of the patient while keeping the jaws parallel to the shaft 230. Inside the abdominal cavity, the jaws may be rotated to a particular angle with respect to the shaft 230, and the jaws can be made to clamp the suture site. Afterwards, a needle may be passed through channels 215, 225 formed in the jaws, whereby the suture site may be sutured.

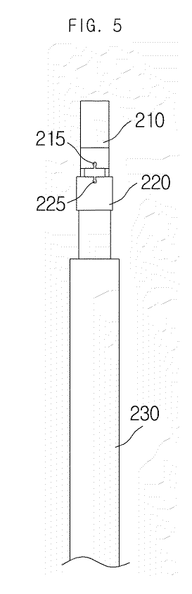

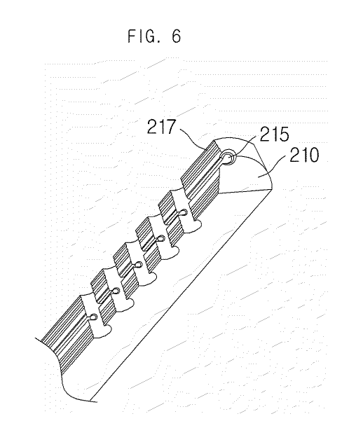

FIG. 5 is a partial side view of one end of a jaw as seen from direction (A) in FIG. 2, while FIG. 6 is a partial perspective view of the other end of the jaw illustrated in FIG. 2. In each of the first jaw 210 and second jaw 220, a channel 215, 225 may be formed that passes through both ends. A needle (not shown) may be inserted through the channels 215, 225 to suture the suture site. Grooves 217 may be formed in the opposing surfaces of the first jaw 210 and second jaw 220, to allow a stronger clamp on the suture site.

The channel 215, 225 can be shaped as a funnel, with the opening having a decreasing inner diameter towards the inside. As this funnel-shaped opening has a wide insertion area, a slightly crooked needle may easily enter the opening, and consequently, the needle can be readily inserted into the channel 215, 225.

In FIG. 6, a multiple number of trenches are illustrated that are formed in the opposing surfaces of the first jaw 210 and second jaw 220, in orthogonal directions to the grooves 217. The flesh of the suture site may enter these trenches, to be stitched by the needle. Multiple openings may be formed, as the channels 215, 225 are disconnected by the multiple trenches, and these multiple openings can also be shaped as funnels. Hence, a needle inserted in an opening at one end of a channel 215, 225 can readily pass through the channel 215, 225, until it emerges through the opening at the other end.

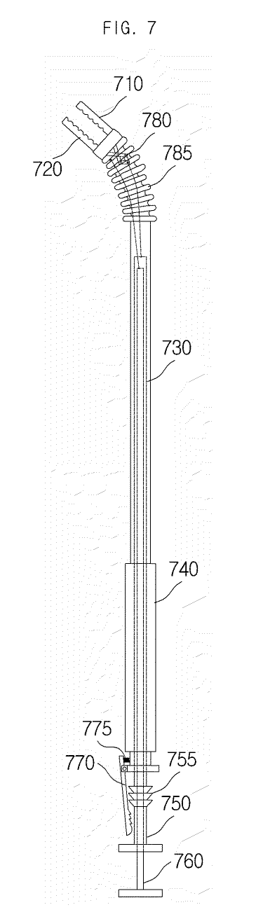

FIG. 7 is a front view of a PSI for laparoscopic surgery according to a second disclosed embodiment of the invention. Illustrated in FIG. 7 are a first jaw 710, a second jaw 720, a shaft 730, a grip 740, a first rod 750, detents 755, a second rod 760, a stopper 770, a spring 775, a scissor link 780, and a bending part 785. The following descriptions will focus mainly on differences from the embodiment described above.

The first jaw 710 and the second jaw 720 may be parallel to each other, and each may have one end coupled to the scissor link 780. The scissor link 780 may be a scissors-type link that is increased in breadth when shortened in the lengthwise direction and decreased in breadth when elongated in the lengthwise direction. The scissor link 780 may have one end coupled to the first rod 750, to be controlled by the first rod 750. That is, the scissor link 780 may be varied in breadth, as its length is varied according to the movement of the first rod 750, and thus the distance between the first jaw 710 and second jaw 720 may also be varied.

The first rod 750 can be connected with the scissor link 780 directly or by way of a wire. FIG. 7 illustrates a coupling relation that uses a wire.

The scissor link 780 and the first jaw 710 and second jaw 720 can be mate-coupled along trenches that are formed at the coupling portions in directions orthogonal to the lengthwise direction of the shaft 730. That is, the first jaw 710 and second jaw 720 can be mate-coupled at the portions of coupling with the shaft 730 in such a way that the length of the scissor link 780 may be changed, with respect to the coupling portions of the scissor link 780 and the first jaw 710 and second jaw 720, while at the same time, the distance between the first jaw 710 and second jaw 720 may be adjusted. Of course, it is obvious that the embodiment can be implemented using a form of coupling other than a mate-coupling.

The bending part 785 may be equipped on one end of the shaft 730 and may be capable of bending while holding the scissor link 780. That is, since the bending part 785 can be bent in correspondence to user manipulation, the user can conveniently perform the suturing, by having the bending part 785 point towards the same direction as the lengthwise direction of the shaft 730 when it is being inserted into the abdominal cavity, and then curving the bending part 785 to a direction convenient for suturing after the jaws have been inserted into the abdominal cavity.

The curving angle of the bending part 785 may be controlled by the operation of the second rod 760, and for this, the bending part 785 and the second rod 760 can be connected to each other by a wire. For example, the bending part 785 can be such that tends to point towards the same direction as the lengthwise direction of the shaft 730 due to an elastic force, and a wire can be connected to a wall inside the bending part 785. In this case, pulling down on the second rod 760 may cause the bending part 785 to curve in one direction due to the tension of the wire. The user can have the jaws face the suture site by rotating the whole shaft 730 about an axis following the lengthwise direction. Of course, various other mechanisms for bending the bending part 785 using wires can be applied to this embodiment.

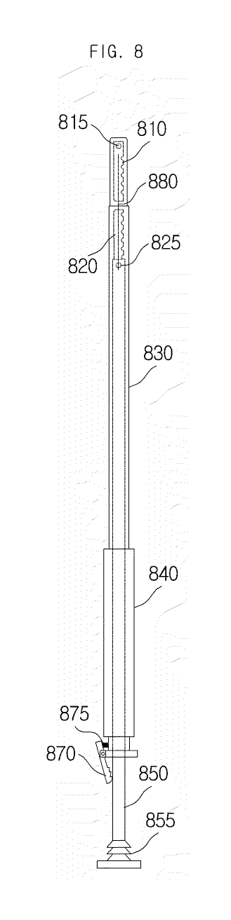

FIG. 8 through FIG. 10 are front views of a PSI for laparoscopic surgery according to a third disclosed embodiment of the invention, for various modes of use. Illustrated in FIGS. 8 to 10 are a first jaw 810, a first hinge axis 815, a second jaw 820, a second hinge axis 825, a shaft 830, a grip 840, a main rod 850, detents 855, a stopper 870, a spring 875, and a rotation member 880. The following descriptions will focus mainly on differences from the embodiments described above.

The first jaw 810 may be rotatably coupled to the first hinge axis 815 provided on the shaft 830, and the second jaw 220 may be coupled to the second hinge axis 825 provided on one end of the main rod 850. The first jaw 810 and the one end of the main rod 850 may be coupled by the rotation member 880. The rotation member 880 may be a member that provides a rotational force to the first jaw 810 and can be an elastic member, such as a spring and a rubber band, which applies an elastic force.

The first jaw 810 may receive a rotational force in a clockwise direction about the first hinge axis 815 from another elastic member, such as an elastic spring, equipped on the first hinge axis 815. Also, the second jaw 820 may receive a rotational force in a counter-clockwise direction about the second hinge axis 825 from an elastic member equipped on the second hinge axis 825.

If the main rod 850 is pulled down to increase the distance between the first jaw 810 and the main rod 850, the first jaw 810 and the second jaw 820 may both rotate to be parallel to the lengthwise direction of the shaft 830 and may be held inside the shaft 830. Thus, the main rod 850 can rotate the first jaw 810 and second jaw 820 while moving linearly.

The first jaw 810 may receive a rotational force in a counter-clockwise direction about the first hinge axis 815 from the rotation member 880, which may be coupled with a particular distance from the first hinge axis 815, and the first jaw 810 may thus rotate to be parallel to the lengthwise direction of the shaft 830. Also, the second jaw 820, as it is pulled downward by the main rod 850, may be rotated by the inner wall of the shaft 830 to become parallel to the lengthwise direction of the shaft 830. In the example shown in FIG. 8, the walls on either side of the shaft 830 where the first jaw 810 is located are open.

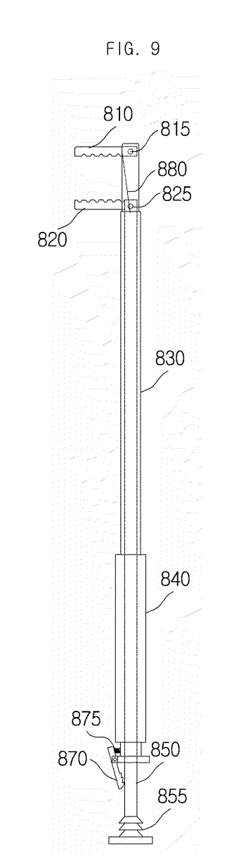

In FIG. 9, an example is illustrated in which the first jaw 810 and second jaw 820 are rotated to protrude out of the shaft 830. When the main rod 850 is moved up, the rotational force applied by the elastic member equipped on the first hinge axis 815 may become greater than the rotational force applied by the rotation member 880, so that the first jaw 810 may rotate clockwise, and its lengthwise direction may not be parallel to the lengthwise direction of the shaft 830. Also, when the main rod 850 is moved up, the second jaw 820 may move to the end of the shaft 830 where the left wall is open, and may thus protrude outwards of the shaft 830 due to the rotational force applied by the elastic member equipped on the second hinge axis 825. In this case, detents (not shown) can be formed on the paths of rotation such that the first jaw 810 and second jaw 820 may be parallel to each other.

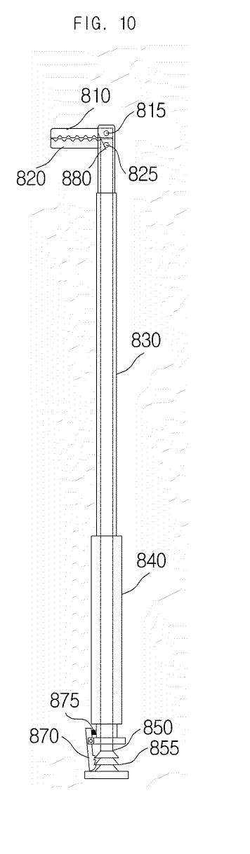

In the example shown in FIG. 10, the first jaw 810 and second jaw 820 may clench together as the main rod 850 moves upwards. The operation of the main rod 850 may be stopped at the lower end by the stopper 870.

While the foregoing descriptions disclose a structure in which the first jaw 810 is rotated by the rotation member 880, the invention is not thus limited. For example, the first jaw 810 can also be rotated by a movement of a separate, second rod, which couples with the first jaw 810, as described above.

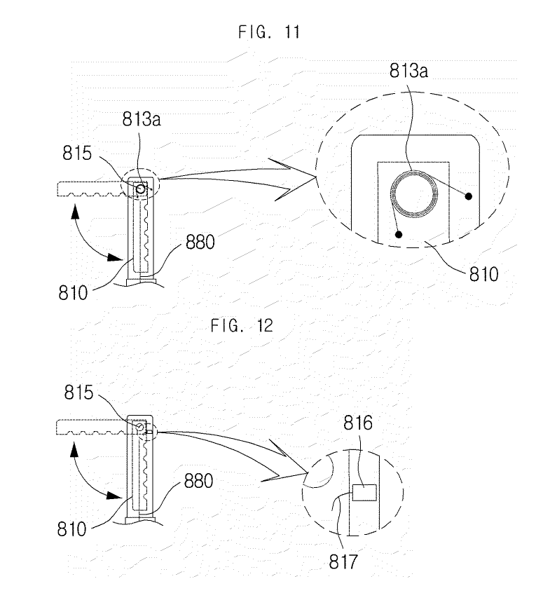

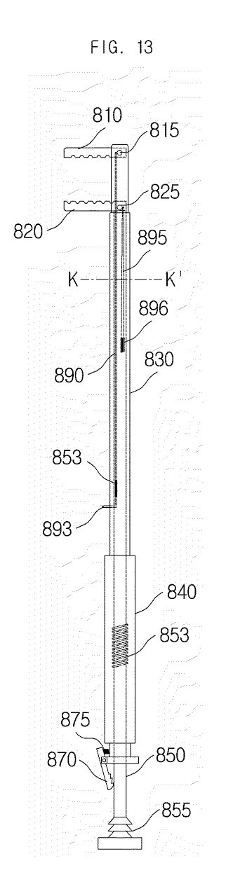

FIG. 11 and FIG. 12 are partially magnified views of a PSI for laparoscopic surgery according to embodiments of the invention.

In the example illustrated in FIG. 11, V-springs 813a, 813b are used as the elastic members described above, as members for rotating the first jaw 810 and second jaw 820 about their respective rotational axes. The V-spring 813a may have its center coupled to the first hinge axis 815, with one end coupled with the first jaw 810 and the other end coupled to the shaft 830, so that the first jaw 810 can be rotated clockwise about the first hinge axis 815 due to the force exerted by the two ends tending to spread apart. Also, the V-spring 813b may have its center coupled to the second hinge axis 825, one end coupled with the second jaw 820, and the other end coupled to the main rod 850, so that the second jaw 820 may be rotated counter-clockwise about the second hinge axis 825 due to the force exerted by the two ends tending to spread apart. Thus, when the main rod 850 is moved in the direction of the first jaw 810 as described above, the first jaw 810 may rotate clockwise about the first hinge axis 815, and the second jaw 820 may rotate counter-clockwise about the second hinge axis 825, to protrude outside of the shaft 830. Of course, various other types of spring, such as a helical spring (coil), can be applied to this embodiment.

In the example illustrated in FIG. 12, a flat spring 817 is used as the elastic member described above, as a member for rotating the first jaw 810. The flat spring 817 can be formed by one or more elastic plates. The flat spring 817 may have one end coupled to the first jaw 810 adjacent to the first hinge axis 815, and the other end coupled to the shaft 830 or to a separate support 816 secured to the shaft 830, so that the first jaw 810 can be rotated in a clockwise direction about the first hinge axis 815 due to the restoring force of the spring. While an example is illustrated in which a V-spring is coupled to the second jaw 820, the second jaw 820 can also be rotated by coupling a flat spring, as described above. Of course, various other types and structures of springs can be applied to this embodiment.

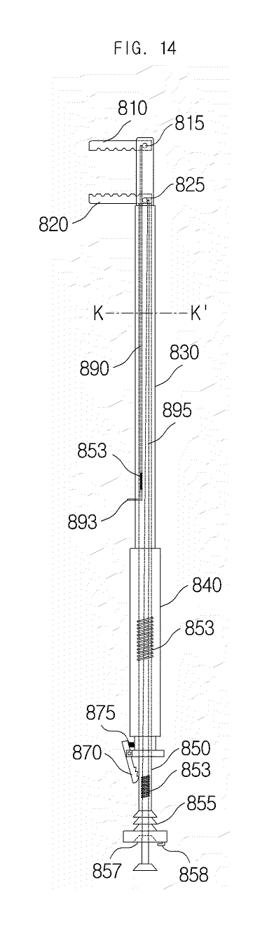

FIG. 13 is a front view of a PSI for laparoscopic surgery according to a fourth disclosed embodiment of the invention. Illustrated in FIG. 13 are a first jaw 810, a first hinge axis 815, a second jaw 820, a second hinge axis 825, a shaft 830, a grip 840, a main rod 850, a spring 853, detents 855, a stopper 870, a spring 875, a first rotation rod 890, a second rotation rod 895, an elastic member 896, and a lever 893. The following descriptions will focus mainly on differences from the above.

In this embodiment, the first jaw 810 may be rotated using the first rotation rod 890, without a separate rotation member 880 coupled to the first jaw 810 and the main rod, 850, and the second jaw 820 may be rotated using the second rotation rod 895.

The first rotation rod 890 may have one end coupled to the first jaw 810 at a particular distance from the first hinge axis 815, while the other end may be equipped with a lever 893. The lever 893 may be formed protruding from a side of the shaft 830, and its location may be specified by securing the lever 893 to a stopper, a latch, or a separate indentation. By moving the lever 893 along the axial direction of the shaft 830, the user may rotate the first jaw 810 coupled to one end of the first rotation rod 890. For example, if one end of the first rotation rod 890 is coupled to the left of the first hinge axis 815 illustrated in FIG. 13, the first jaw 810 may be rotated about the first hinge axis 815 in a counter-clockwise direction when the first rotation rod 890 is moved downwards, while the first jaw 810 may be rotated about the first hinge axis 815 in a clockwise direction when the first rotation rod 890 is moved upwards.

Also, the second rotation rod 895 may have one end coupled to the second jaw 820 at a particular distance from the second hinge axis 825, with one side coupled to the elastic member 896, so that the second jaw 820 can be rotated in one direction by the elastic force of the elastic member 896. The elastic member 896 can be coupled to various positions, such as the far end or a middle side of the second rotation rod 895, to apply an elastic force on the second rotation rod 895. The elastic member 896 is not limited to a particular shape or structure, as long as it is a member for applying an elastic force on the second rotation rod 895, and the elastic member 896 can be, for example, a helical spring.

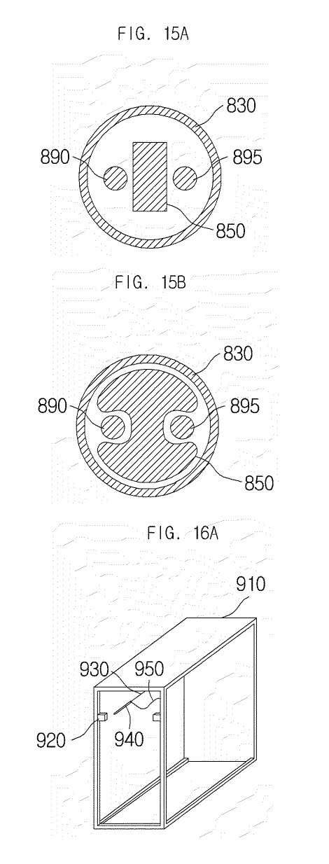

The elastic member 896 may be securely held on one side of the main rod 850, on the inside, for example, and may apply an elastic force to pull or push the second rotation rod 895 towards the second jaw 820, allowing the second jaw 820 to rotate in one direction. For example, if one end of the second rotation rod 895 is coupled to the right of the second hinge axis 825, as illustrated in FIG. 14, the second rotation rod 895 may push the second jaw 820, due to the elastic force of the elastic member 896 coupled to the other end, so that the second jaw 820 may continuously receive a force that rotates the second hinge axis 825 in a counter-clockwise direction. Also, if one end of the second rotation rod 895 is coupled to the left of the second hinge axis 825 illustrated FIG. 14, the second rotation rod 895 can pull the second jaw 820, according to the elastic force of the elastic member 896 coupled to the other end, in which case the second jaw 820 may continuously receive a force that rotates the second hinge axis 825 in a counter-clockwise direction.

Therefore, when the main rod 850 is moved so that the second jaw 820 coupled to the main rod 850 may be opened out of the shaft 830, the elastic force of the elastic member 896 may be transferred to the second jaw 820, which may then rotate counter-clockwise to be arranged orthogonally to the extending direction of the shaft 830.

A portion of the first rotation rod 890 and/or main rod 850 can be formed as a spring 853. The spring 853 can be a typical helical spring and can serve to absorb and buffer impact energy. By forming a portion of the first rotation rod 890 and/or main rod 850 as a spring, as described above, the spring 853 may buffer the impact and prevent damage to the suture site when the thickness of the suture site is varied, and a constant pressure for clamping can be maintained.

FIG. 14 is a front view of a PSI for laparoscopic surgery according to a fifth disclosed embodiment of the invention. Illustrated in FIG. 14 are a first jaw 810, a first hinge axis 815, a second jaw 820, a second hinge axis 825, a shaft 830, a grip 840, a main rod 850, a spring 853, detents 855, an indentation 857, a securing element 858, a stopper 870, a spring 875, a first rotation rod 890, a second rotation rod 895, and a lever 893. The following descriptions will focus mainly on differences from the above.

In the example shown in FIG. 14, the first jaw 810 may be rotated using the first rotation rod 890, without a separate rotation member 880 coupled to the first jaw 810 and the main rod 850, and the second jaw 820 may be rotated using the second rotation rod 895, which extends to one end of the main rod 850.

The first rotation rod 890 may have one end coupled to the first jaw 810 at a particular distance from the first hinge axis 815, while the other end may be equipped with a lever 893. The lever 893 may be formed protruding from a side of the shaft 830, and its location may be specified by securing the lever 893 to a stopper, a latch, or a separate indentation. By moving the lever 893 along the axial direction of the shaft 830, the user may rotate the first jaw 810 coupled to one end of the first rotation rod 890. For example, if one end of the first rotation rod 890 is coupled to the left of the first hinge axis 815 illustrated in FIG. 14, the first jaw 810 may be rotated about the first hinge axis 815 in a counter-clockwise direction when the first rotation rod 890 is moved downwards, while the first jaw 810 may be rotated about the first hinge axis 815 in a clockwise direction when the first rotation rod 890 is moved upwards.

Also, the second rotation rod 895 may have one end coupled to the second jaw 820 at a particular distance from the second hinge axis 825, to operate in a manner similar to that of the first rotation rod 890 described above and thereby rotate the second jaw 820 in. For example, if one end of the second rotation rod 895 is coupled to the right of the second hinge axis 825 illustrated in FIG. 14, the second jaw 820 may be rotated about the second hinge axis 825 in a clockwise direction when the second rotation rod 895 is moved downwards, while the second jaw 820 may be rotated about the second hinge axis 825 in a counter-clockwise direction when the second rotation rod 895 is moved upwards.

The second rotation rod 895 can be held inside the main rod 850, and in this case, if the other end of the second rotation rod 895 is coupled with the main rod 850, the other end of the second rotation rod 895 can be inserted in the indentation 857 formed in the portion where it couples with the main rod 850. The indentation 857 of the main rod 850 can be formed in correspondence with the shape of the other end of the second rotation rod 895. When the other end of the second rotation rod 895 is inserted in the indentation 857 of the main rod 850, the securing element 858 can be fastened to the other end of the second rotation rod 895 to stop the movement of the second rotation rod 895. The securing element 858 may be an element that is movable along a direction orthogonal to the extending direction of the second rotation rod 895, and can be implemented and expressed as various structures, such as a stopper, latch, etc.

Also, a portion of the first rotation rod 890 and/or second rotation rod 895 can be formed as a spring 853. The spring 853 may be a helical spring and can serve to absorb and buffer impact energy. By forming a portion of the first rotation rod 890 and/or second rotation rod 895 as a spring, as described above, the spring 853 may buffer the impact and prevent damage to the suture site when the thickness of the suture site is varied, and a constant pressure for clamping can be maintained. A portion of the main rod 850 can also be formed as a spring 853.

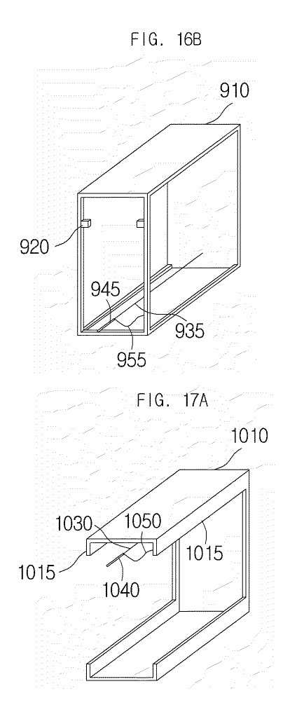

The shape by which the main rod 850, first rotation rod 890, and second rotation rod 895 are held inside the shaft 830 can be implemented in various ways. Referring to FIG. 15A and FIG. 15B, which are cross-sectional views across the lines K-K′ in FIG. 13 and FIG. 14, the shaft 830 can be shaped as a generally hollow tube, and the main rod 850, first rotation rod 890, and second rotation rod 895 can be held inside the shaft 830 (FIG. 15A). Also, referring to FIG. 15B, the shaft 830 can be shaped as a generally hollow tube, and the main rod 850 can include separate trenches extending along the axial direction to hold the first rotation rod 890 and second rotation rod 895, while the first rotation rod 890 and the second rotation rod 895 can each be held in a trench to move along the axial direction of the shaft 830.

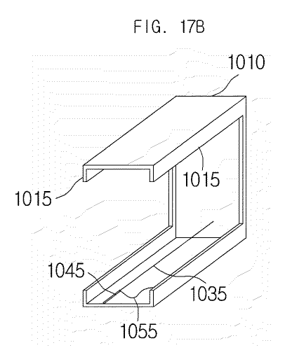

FIG. 16A and FIG. 16B are perspective views of cabinets for PSI's for laparoscopic surgery according to another embodiment of the invention. Illustrated in FIGS. 16A and 16B are a frame 910, protruding parts 920, a first support part 930, a second support part 935, a first needle 940, a second needle 945, a first thread 950, and a second thread 955.

In an attempt to resolve the problem of difficulty in inserting a needle into the jaws described above, this embodiment provides a cabinet that corresponds with the size of the jaws and enables a needle to be inserted into the jaws at the same time as the jaws are inserted. While FIGS. 16A and 16B respectively illustrate examples in which the first needle 940 and the second needle 945 are provided separately, the invention is not limited to this structure. It is obvious, for example, that the first needle 940 and second needle 945 can both be coupled to a single frame 910 with a particular distance in-between.

The frame 910 may be a scaffold into which the jaws may be inserted and may be formed in correspondence with the size of the jaws. Since the jaws are to be inserted into the cabinet while clamping the suture site, the cabinet according to this embodiment can be fabricated correspondingly after measuring the size of the jaws clamping the suture site. The frame 910 can be formed simply as a scaffold for maintaining its shape, or the frame 910 can be formed with its sides blocked other than the opening through which the jaws are inserted.

The first support part 930, first needle 940, and first thread 950 may correspond with the first jaw 110, 210, 710, 810, while the second support part 935, second needle 945, and second thread 955 may correspond with the second jaw 120, 220, 720, 820. In cases where the first jaw 210 is inserted into the frame 910, the first needle 940 facing the lengthwise direction of the first channel 215 may be inserted into the first channel 215, and thus the first thread 950 may stitch the suture site. In this way, the user may resolve the difficulty of inserting only the first needle 940 into the first channel 215 as described above.

The first support part 930 may be coupled to the frame 910 to support the first needle 940, and when the first jaw 210 is inserted in the frame 910, may apply the force for inserting the first needle 940 into the first channel 215. To this end, the first support part 930 may be formed orthogonally to the direction in which the first jaw 210 is inserted and moved, or as in the illustrated example, may extend along a parallel direction, to support the first needle 940. In the latter case, the first support part 930 can be coupled to the rear surface of the frame 910 facing the opening through which the first jaw 210 is inserted.

Also, in the former case, a support-part through-channel (not shown) can be formed in the first jaw 210 through which the first support part 930 may pass. That is, since the first support part 930 may hinder the movement of the first jaw 210 if the first support part 930 is installed extending along a direction orthogonal to the direction in which the first jaw 210 is moved, a support-part through-channel can be formed in the first jaw 210 to resolve this issue.

As in the illustrated example, the first support part 930 can be provided at the opening through which the first jaw 210 is inserted or at an arbitrary point along the direction in which the first jaw 210 is inserted. The first needle 940 can also be positioned at the opening through which the first jaw 210 is inserted or at an arbitrary point along the direction in which the first jaw 210 is inserted.

The protruding parts 920 may be coupled to the frame 910 and may be inserted in correspondence with the trenches formed in the jaw. The protruding parts 920 allow the first needle 940 to be inserted in the first channel 215 more accurately. That is, groove-like trenches can be formed in the first jaw 210 with their relative distances to the first channel 215 calculated beforehand, and the first jaw 210 can be inserted into the frame 910 by coupling the trenches with the protruding parts 920, so that the first needle 940 may be inserted into the first channel 215 accurately. There is no particular limit to the number of protruding parts 920, and FIGS. 16A and 16B each illustrate examples that include two.

Of course, the above descriptions can also be applied to the second support part 935, second needle 945, second thread 955, and second jaw 120. Furthermore, the number of support parts, including the first support part 930 and second support part 935, can be determined in correspondence to the number of channels formed in the jaws.

Also, the cabinet can be manipulated by the tongs of a surgical instrument or can be integrated with an instrument dedicated to its use.

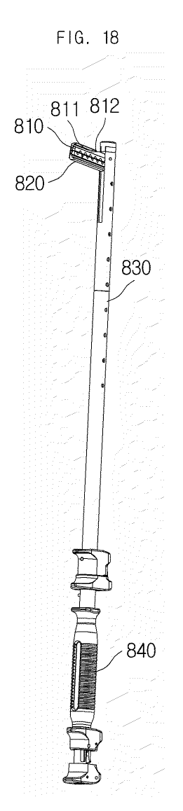

FIG. 17A and FIG. 17B are perspective views of cabinets for PSI's for laparoscopic surgery according to yet another embodiment of the invention. Illustrated in FIGS. 17A and 17B are a frame 1010, cabinet sides 1015, a first support part 1030, a second support part 1035, a first needle 1040, a second needle 1045, a first thread 1050, and a second thread 1055. The following descriptions will focus mainly on differences from the embodiments described above.

A feature of this embodiment is that, since the jaws would be clamping the suture site when the needle is to be inserted in the jaws, the cabinet to which the PSI for laparoscopic surgery is applied has its sides opened, so that the jaws may readily be inserted into the frame 1010. That is, both sides of the cabinet may be open, so that the jaws may readily be inserted into the frame 1010, even when portions of the suture site protrude at the sides.

The cabinet sides 1015 may extend a particular length from the upper portion, and these parts may allow easier insertion of the jaws. The distance between the cabinet sides 1015 at the upper portion can be formed with the same breadth as that of the jaws, so that the cabinet sides 1015 may serve as the protruding parts 920 described above. Of course, this embodiment can also include additional protruding parts 920.

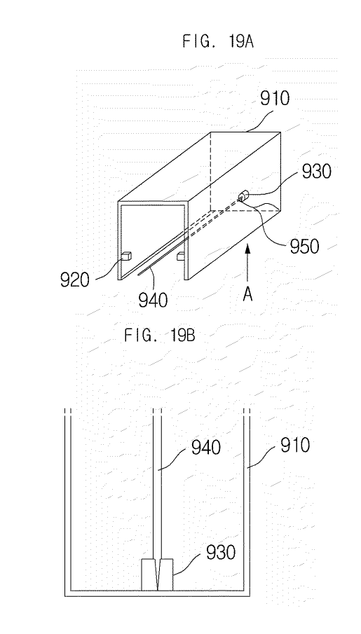

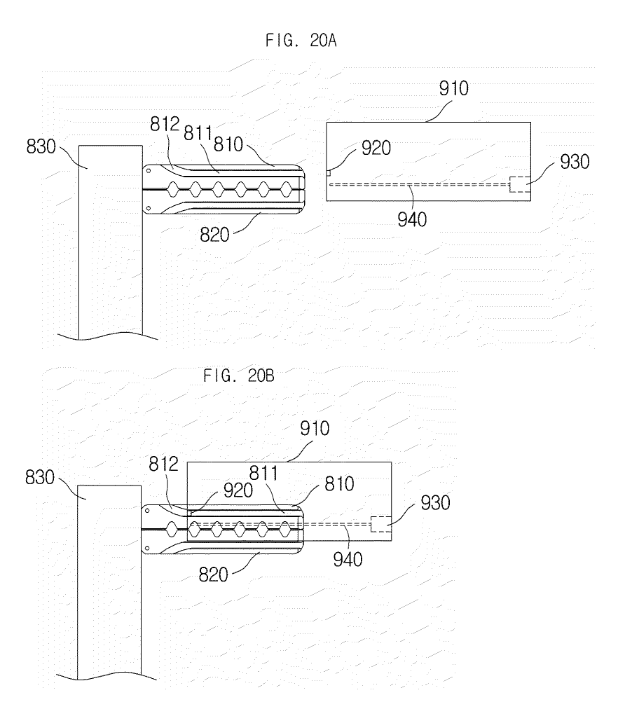

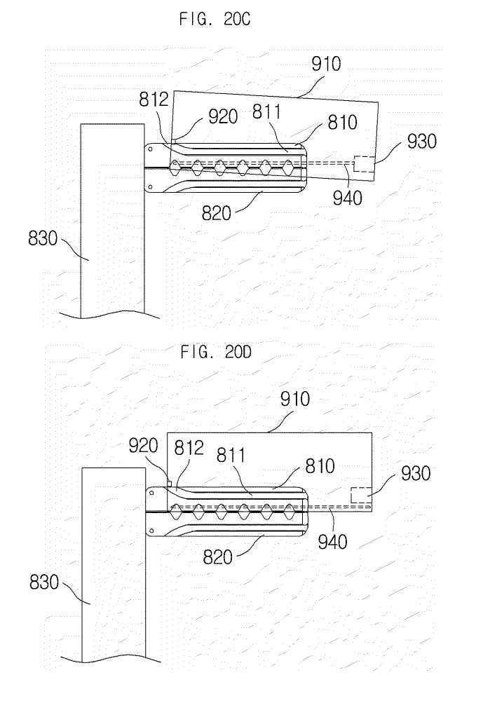

FIG. 18 is a perspective view of a PSI for laparoscopic surgery according to an eighth disclosed embodiment of the invention, FIG. 19A is a perspective view of a cabinet for a PSI for laparoscopic surgery for inserting a needle into the PSI for laparoscopic surgery, and FIG. 19B is a partial rear view of the cabinet of FIG. 19A as seen from direction “A”, while FIG. 20A through FIG. 20D illustrate a process of inserting a needle in the PSI for laparoscopic surgery. Illustrated in FIGS. 18 to 20D are a first jaw 810, trenches 811, angled parts 812, a second jaw 820, a shaft 830, a grip 840, a frame 910, protruding parts 920, a first support part 930, a first needle 940, and a first thread 950. While the following descriptions will mainly be provided with regards the form, function, and operation of the first jaw 810, it is obvious that the descriptions can also apply to the second jaw 820. The following descriptions will focus mainly on differences from the embodiments described above.

A feature of this embodiment is to form a trench having a particular slope in the PSI for laparoscopic surgery, and form the protruding part, support part, and frame of the cabinet correspondingly, in order that the needle may readily be inserted into the PSI for laparoscopic surgery. That is, with a PSI for laparoscopic surgery according to this embodiment, when the user pushes the jaws in one direction, the PSI may be detached from the cabinet for the PSI while the needle is inserted in the jaws.

In the first jaw 810 and/or second jaw 820, groove-like trenches 811 may be formed, in which the protruding parts 920 formed on the cabinet for the PSI for laparoscopic surgery may be held. As in the illustrated example, the trenches 811 may be formed to be generally parallel to the extending directions of the first jaw 810 and second jaw 820, respectively, and therefore the user can push the first jaw 810 and second jaw 820 along the extending direction, with the protruding parts 920 inserted in the trenches 811, so that the first needle 940 may be inserted in the first jaw 810. Although only the first needle 940 is illustrated in the drawings, it is obvious that a second needle 945 may additionally be included, which may be inserted into the channel of the second jaw 820, as described above.

Referring to FIG. 19A, the frame 910 can be shaped as a “C”, having one side open, and in this case, the first jaw 810 and second jaw 820, which may be inserted into the cabinet such that a needle is inserted in each channel, can be detached from the cabinet through the opened side. The protruding parts 920 may be formed in locations corresponding to the trenches 811 of the first jaw 810, and the first needle 940 may be positioned such that it can be inserted into the channel formed in the first jaw 810. Thus, as the protruding parts 920 are inserted into the trenches 811 of the first jaw 810, the first needle 940 can be inserted into the channel formed in the first jaw 810.

The first support part 930 may be formed on the surface opposite the opening of the frame 910 through which the first jaw 810 is inserted, and may support the first needle 940. For example, the first needle 940 may be supported while lodged in the first support part 930. The first support part 930 may be formed from a flexible material such that the first needle 940 can be detached from the first support part 930 when the first needle 940 receives a force in a direction orthogonal to its extending direction. To this end, a side gap can be formed in the first support part 930 that can provide a detachment path when the first needle 940 receives the above force. For example, as illustrated in FIG. 19A and FIG. 19B, the first support part 930 can be shaped as a cylinder, a cylindroid (including a partial cylindroid), a prism, etc., and a hole may be formed in a center portion, into which the first needle 940 can be inserted, while a gap may be formed in the side. Thus, when a force is applied to the first needle 940 inserted in the first support part 930 in a direction orthogonal to its extending direction, the first needle 940 can be detached from the first support part 930 through the gap.

Referring to FIGS. 20A to 20D, the first jaw 810 may include trenches 811 formed parallel to the extending direction of the first jaw 810, while an angled part 812 having a particular slope with respect to the parallel direction may be formed in one end of a trench 811. Here, the slope can be 0 to 90 degrees, and the angled parts 812 can be formed in various shapes, such as straight or curved lines, etc. Referring to FIG. 20A, the protruding part 920 of the cabinet may correspond with the location of the trench 811, and the first needle 940 may correspond with the location of a channel of the first jaw 810. The protruding part 920 of the cabinet may be inserted in and moved along the trench 811, whereby the movement path of the cabinet may be specified (see FIG. 20B). As the protruding part 920 is moved along the angled part 812, the opening of the cabinet through which the first jaw 810 is inserted may be increased in height relative to the first jaw 810, whereas the height of the first support part 930 may remain constant until the first needle 940 is detached, and therefore the cabinet may be slanted, with the left side of the cabinet higher than the other, as illustrated in FIG. 20C. Afterwards, the first needle 940 may continue to receive a force in a direction orthogonal to its extending direction, and when the force exceeds a detachment threshold, the first needle 940 may be detached from the first support part 930 (see FIG. 20D).

Thus, according to this embodiment, the user can apply a pushing force in one direction to insert a needle into a jaw and withdraw the jaw from the cabinet at the same time.

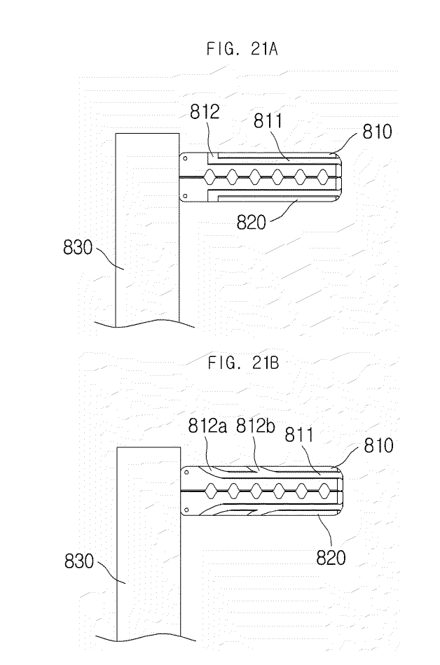

FIG. 21A and FIG. 21B are partial side views of a PSI for laparoscopic surgery according to another embodiment of the invention. In the example shown in FIG. 21A, the slope of the angled part 812 described above with respect to the extending direction of the first jaw 810 is 90 degrees. In this case, the user may push the first jaw 810 into the cabinet until the protruding part 920 reaches the angled part 812, and then move the first jaw 810 in a direction parallel to the angled part 812, to detach the first jaw 810 from the cabinet.

Referring to FIG. 21B, the angled part 812 described above can be included in multiple numbers. Since the positions of the angled parts 812 can be determined by the length of the first needle 940 and the degree to which it protrudes, etc., the assembly can be applied in various ways to various cabinet structures, conditions, environments, etc., by using multiple angled parts 812 in different positions.

Those of ordinary skill in the art will understand that various changes and modifications can be made to the invention without departing from the spirit and scope of the invention as defined by the appended claims below.

What is claimed is:

1. A PSI for laparoscopic surgery, the instrument comprising:
a first jaw;
a second jaw configured to clamp a suture site in cooperation with the first jaw;
a first rod coupled to one end of the first jaw and configured to rotate the first jaw such that the suture site is clamped, the first rod held inside a shaft; and
a second rod coupled to one end of the second jaw and configured to rotate the second jaw such that the second jaw faces an extending direction of the shaft,
wherein a diameter of the jaws when the first jaw and the second jaw are clenched is smaller than a diameter of the shaft.

2. The PSI for laparoscopic surgery according to claim 1, wherein the first jaw and the second jaw are inserted inside the shaft by a linear movement of the second rod.

3. A PSI for laparoscopic surgery, the instrument comprising:
a set of jaws including a first jaw and a second jaw configured to clamp a suture site;
a scissor link coupled to one end of each of the first jaw and the second jaw for adjusting a distance between the first jaw and the second jaw; and
a first rod coupled to one end of the scissor link and configured to control the scissor link such that the suture site is clamped.

4. The PSI for laparoscopic surgery according to claim 3, further comprising:
a shaft configured to hold the first rod and equipped on one end thereof with a bending part, the bending part capable of bending.

5. The PSI for laparoscopic surgery according to claim 4, further comprising:
a second rod coupled to the bending part and configured to adjust a bending angle of the bending part.

6. The PSI for laparoscopic surgery according to claim 1, wherein the second rod is held inside the first rod.

7. The PSI for laparoscopic surgery according to claim 1, further comprising:
a stopper coupled to one end of the shaft and configured to be caught on a detent formed on one end of the first rod such that the first rod is stopped.

8. The PSI for laparoscopic surgery according to claim 5, wherein the first rod and the second rod are configured to move linearly.

9. The PSI for laparoscopic surgery according to claim 1, wherein a portion of the first rod is formed as a spring.

10. A PSI for laparoscopic surgery, the instrument comprising:
a first jaw configured to be movable and rotatable by way of a first hinge axis;
a second jaw configured to clamp a suture site in cooperation with the first jaw, the second jaw configured to be movable and rotatable by way of a second hinge axis;
a shaft holding the first jaw and the second jaw; and
a main rod coupled to the second hinge axis and configured to move the second hinge axis inside the shaft.

11. The PSI for laparoscopic surgery according to claim 10, further comprising:
a rotation member coupled with the first jaw and configured to apply a rotational force on the first jaw.

12. The PSI for laparoscopic surgery according to claim 10, further comprising:
an elastic member coupled to one or more of the first hinge axis and the second hinge axis and configured to rotate the first jaw or the second jaw.

13. The PSI for laparoscopic surgery according to claim 10, further comprising:
a first rotation rod having one end thereof coupled to the first jaw, the first rotation rod held inside the shaft and configured to rotate the first jaw; and
a second rotation rod having one end thereof coupled to the second jaw, the second rotation rod held inside the shaft and configured to rotate the second jaw.

14. The PSI for laparoscopic surgery according to claim 13, further comprising:
an elastic member coupled to one side of the second rotation rod and configured to apply an elastic force on the second rotation rod so as to rotate the second jaw in one direction.

15. The PSI for laparoscopic surgery according to claim 13, wherein the shaft is shaped as a hollow tube, and the first rotation rod and the second rotation rod are held inside the shaft.

16. The PSI for laparoscopic surgery according to claim 13, wherein the main rod has a trench formed therein extending along an axial direction of the main rod, and the first rotation rod and the second rotation rod are held in the trench.

17. The PSI for laparoscopic surgery according to claim 13, wherein the other end of the first rotation rod is coupled to a lever protruding from a side surface of the shaft.

18. The PSI for laparoscopic surgery according to claim 13, wherein one end of the main rod has an indentation formed therein, and the other end of the second rotation rod is configured to fit into the indentation.

19. The PSI for laparoscopic surgery according to claim 10, further comprising:
a stopper coupled to one end of the shaft and configured to be caught on a detent formed on one end of the first rod such that the first rod is stopped.

20. The PSI for laparoscopic surgery according to claim 10, wherein a portion of the main rod is formed as a spring.

21. The PSI for laparoscopic surgery according to claim 1, wherein a channel formed through both ends of one or more of the first jaw and the second jaw and having a needle inserted therein has an opening thereof shaped as a funnel.

22. The PSI for laparoscopic surgery according to claim 4, wherein a diameter of the jaws when the first jaw and the second jaw are clenched is smaller than a diameter of the shaft.

23. A cabinet for a PSI for laparoscopic surgery configured to insert a needle into a jaw having a channel passing through both ends thereof, the cabinet comprising:
a frame having the jaw inserted therein; and
a support part coupled to the frame and configured to support the needle in correspondence with an opening of the channel when one end of the jaw is inserted.

24. The cabinet for a PSI for laparoscopic surgery according to claim 23, further comprising:
a protruding part coupled to the frame and configured to be inserted in correspondence with a trench formed in the jaw.

25. The cabinet for a PSI for laparoscopic surgery according to claim 24, wherein the trench formed in the jaw is parallel to an extending direction of the jaw.

26. The cabinet for a PSI for laparoscopic surgery according to claim 24, wherein the trench formed in the jaw includes an angled part slanting by a particular slope with respect to an extending direction of the jaw.

27. The cabinet for a PSI for laparoscopic surgery according to claim 26, wherein the angled part is formed at an end portion of the trench.

28. The cabinet for a PSI for laparoscopic surgery according to claim 23, wherein the cabinet has one or more open sides other than an opening through which the jaw is inserted.

29. The cabinet for a PSI for laparoscopic surgery according to claim 23, wherein the support part is located at a surface opposite an opening through which the jaw is inserted.

30. The cabinet for a PSI for laparoscopic surgery according to claim 29, wherein a side gap is formed in a side of the support part, the side gap configured such that the inserted needle is detached from the support part when a force is applied in a direction orthogonal to an extending direction of the needle.
